(19) United States
(12) Patent Application Publication
(10) Pub. No.: US 2010/0298602 A1
(43) Pub. Date: Nov. 25, 2010

(54) SYSTEMS AND METHODS FOR MICROFLUIDIC CRYSTALLIZATION

(75) Inventors: Mahmooda Sultana, Cambridge, MA (US); Klavs F. Jensen, Lexington, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/468,365

(22) Filed: May 19, 2009

(51) Int. Cl. C07C 229/08; G01N 15/00; C07C 229/06

(52) U.S. Cl. 562/553; 73/61.71; 356/300

(57) ABSTRACT

Systems and methods for crystallization in microfluidic systems are generally described. Many applications require the collection of time-resolved data to determine advantageous conditions for crystallization. The present invention provides tools and related techniques which address this need, as well as a platform for the growth of crystals within microfluidic channels. The systems and methods described herein provide, in one aspect, tools that allow for controlled, stable crystallization of organic materials in microfluidic channels. The invention can interface not only with microfluidic/microscale equipment, but with macroscale equipment to allow for the easy injection of fluids (e.g., fluids containing crystal precursor), extraction of crystals, determination of one or more crystal properties (e.g., crystal size, size distribution among multiple crystals, morphology, etc.), etc.

### FIELD OF INVENTION

Systems and methods relating to crystallization in microfluidic systems are generally described.

### BACKGROUND

Crystallization is the process of forming solid crystals from a precursor such as, for example, a solution, a melt, or a vapor. Traditionally, crystallization has been performed in batch processes that may suffer from non-uniform process conditions such as temperature, important in temperature-driven crystallization, and concentration, important in concentration-driven crystallization. Traditional batch processes may also suffer from poorly controlled mixing of reagents, important in precipitation and antisolvent-driven crystallization. Consequently, crystal growth can vary across the reactors, giving rise to polydisperse crystal size distribution (CSD). This may reduce reproducibility of the crystallization process and increase difficulty in obtaining accurate kinetics data. In addition, batch processes may limit the number of crystallization experiments that may be performed over a given length of time within a particular device. Moreover, changing process parameters in micro batches may require that the entire apparatus be reconfigured, possibly resulting in an entire batch being wasted at once.

Accordingly, improved systems and methods are desired.

### SUMMARY OF THE INVENTION

The embodiments described herein generally relate to systems and method for crystallization in microfluidic systems. The subject matter of the present invention involves, in some cases, interrelated products, alternative solutions to a particular problem, and/or a plurality of different uses of one or more systems and/or articles.

In one aspect, a method is described. In some embodiments, a method of determining crystallization comprises flowing a fluid containing an organic crystal and crystal precursor into a microfluidic channel, determining a first property of the crystal at a first point in the microfluidic channel, and determining a second property of the crystal at a second point in the microfluidic channel.

In some instances, a method of forming crystals comprises flowing a first fluid containing organic crystal seeds into a first microfluidic channel, flowing a second fluid containing a solution of crystal precursor into a second microfluidic channel, and combining the first and second fluids to form a first mixed fluid.

In some embodiments, a method of forming crystals comprises flowing a first fluid containing organic crystal precursor into a microfluidic channel at a first feed inlet, and flowing the first fluid containing organic crystal precursor into the microfluidic channel at a second feed inlet downstream of the first feed inlet.

In some cases, the method comprises determining at least one property of a crystal, comprising a species, in a microfluidic channel, and, based upon the crystal determination step, determining at least one condition for crystallization of the species. The method may further comprise growing crystals comprising the species involving the at least the condition.

In some embodiments, a method of determining particle formation comprises flowing a fluid containing a particle with an aspect ratio of at least about 3:1 and a particle precursor within a microfluidic channel. The method may further comprise determining a first property of the particle at a first point in the microfluidic channel and determining a second property of the particle at a second point in the microfluidic channel. In some embodiments, the determining steps may be performed after the particle is substantially aligned in the direction of fluid flow within the microfluidic channel.

In some aspects; a device is described. The device may comprise, in some embodiments, a primary microfluidic channel having an upstream portion and a downstream portion, wherein fluid flows from the upstream portion to the downstream portion. The device may further comprise a feed section including a first source inlet connectable to a first fluid source, a second source inlet connectable to a second fluid source, and a mixing region in fluid communication with the first and second source inlets, at which fluids from the first and second sources are mixed. The device may further comprise a first channel connecting the mixing region with a first feed inlet to the primary microfluidic channel, for delivery of fluid from the mixing region to the primary microfluidic channel, and a second channel connecting the mixing region with a second feed inlet to the primary microfluidic channel, for delivery of fluid from the mixing region to the primary microfluidic channel.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B include schematic diagrams of devices, according to one set of embodiments;

FIG. 2 includes, according to one set of embodiments, a schematic diagram of a device;

FIGS. 3A-3B include schematic diagrams of crystals, according to one set of embodiments;

FIG. 4 includes a photograph of a channel containing crystals, according to one set of embodiments;

FIGS. 5A-5D include, according to one set of embodiments, schematic diagrams outlining a method of measuring crystal size;

FIG. 6 includes photographs of crystals, according to one set of embodiments;

FIG. 7 includes a schematic diagram of a device, according to one set of embodiments;

FIG. 8 includes a schematic diagram of a crystal, according to one set of embodiments;

FIG. 9 includes a simulated flow profile of fluid flow within a channel, according to one set of embodiments; and

FIG. 10 includes plots of supersaturation as a function of position along a cross-section of a channel for various positions along the length of the channel, according to one set of embodiments.

### DETAILED DESCRIPTION

Systems and methods for crystallization in microfluidic systems are generally described. Many applications require the collection of time-resolved data to determine advantageous conditions for crystallization. The present invention provides tools and related techniques which address this need, as well as a platform for the growth of crystals within microfluidic channels. The systems and methods described herein provide, in one aspect, tools that allow for controlled, stable crystallization of organic materials in microfluidic channels. The invention can interface not only with microfluidic/microscale equipment, but with macroscale equipment to allow for the easy injection of fluids (e.g., fluids containing crystal precursor), extraction of crystals, determination of one or more crystal properties (e.g., crystal size, size distribution among multiple crystals, morphology, etc.), etc.

One challenge in achieving continuous crystallization in microfluidic systems identified in the past is the reduction of the irregular and uncontrolled formation and growth of crystals at the surface of the microfluidic channel. The aggregation of such crystals may ultimately lead to clogging of the microfluidic channel. The systems and methods described herein involve the introduction of reagents to a microfluidic channel in a controlled manner, preventing heterogeneous nucleation and aggregation. For example, in one set of embodiments, controlled crystallization may be achieved by combining a first fluid containing organic crystal seeds and, optionally, organic crystal precursor with a second fluid containing an organic crystal precursor in a microfluidic channel. In some embodiments, the second fluid containing organic crystal precursor may also be introduced into the microfluidic channel at a point downstream of the point where the first and second fluids were originally mixed.

In addition, optical microscopy has been used for the in situ characterization of at least one crystal property, which may be used, for example, to determine crystallization kinetics. Using batch crystallization methods, it may be difficult to accurately measure the size distribution, and thus the growth kinetics, of crystals. The embodiments described herein enable accurate measurement of crystal properties (e.g., size distribution, etc.), and hence accurate determination of crystallization kinetics under continuous flow conditions. In one set of embodiments, a property of a crystal is determined in at least two places within a microfluidic channel, allowing for the determination of a condition for crystallization. The condition may be used, for example, in a subsequent crystallization process. For example, the size of a crystal may be determined at a first point and a second point within a microfluidic channel, and the size measurements may be used to calculate the growth kinetics of the crystal, which may be used to, for example, optimize the growth of the crystal at the macro-scale, as well as control the final size and size distribution of the crystal products.

The systems and methods described herein may be used in a variety of applications that can benefit from the ability to grow crystals in microfluidic channels. For example, a large percentage of all pharmaceutical products are formulated in particulate form, usually in crystalline form. Thus, crystallization is one of the most important unit operations in the pharmaceutical industry. In addition, crystallization may play an important role in the formation of products in the food, pigment, and specialty chemicals industries.

The systems and methods described herein provide several advantages over traditional crystallization methods. For example, continuous operation allows for high-throughput screening of the effects of various process conditions on crystallization. In continuous crystallization experiments, evaporation rate (e.g., of a solvent, of another fluid, etc.), or temperature can be easily varied, enabling one to perform multiple experiments at different temperatures and evaporation rates without having to perform each experiment separately, as done for batch systems. In addition, crystallization conditions such as solvent composition, inhibitor composition and/or concentration, enhancer composition and/or concentration co-crystal composition and/or concentration, precursor composition and/or concentration, impurity composition and/or concentration, or pH of a fluid can be varied by simply changing the flow rate of different feeds, thus facilitating fast screening of the effects of changes in one or more conditions for crystallization. Continuous crystallization also enables the screening of many crystals in a short period of time, thus reducing errors associated with crystal growth rate or dissolution rate dispersion.

In addition, microfluidic systems may include well-defined laminar flow profiles, which allow for the easy determination of flow fields within the channel. Laminar flow may also lead to self-alignment of the crystals, for example, in cases where high aspect-ratio crystals are grown. The self-alignment of the crystals may lead to more accurate measurement of properties such as, for example, crystal size. The short length scale of the microfluidic channel also allows for better control over conditions for crystallization (e.g., temperature, concentration, contact mode of the reagents, etc.) creating substantially uniform process conditions across the reactor channel in some cases. Thus, the systems and methods described herein have the potential to generate a more uniform size distribution of crystals and more accurate kinetics data. Moreover, microfluidic systems decrease waste, provide safety advantages, and require only small amounts of reactants, which is beneficial when dealing with expensive materials such as pharmaceutical drugs.

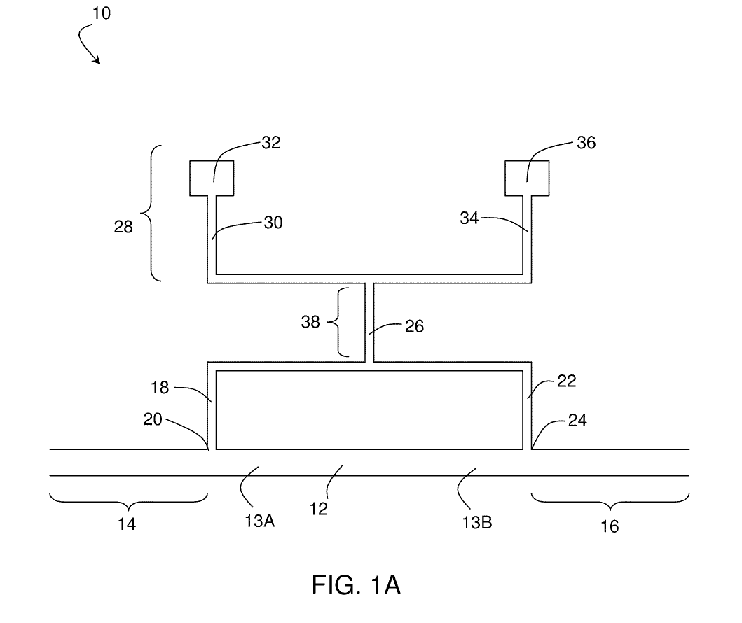

In one set of embodiments, systems and methods related to forming crystals are described. FIG. 1A includes a schematic illustration of a device 10 according to one set of embodiments. Device 10 comprises a primary fluidic channel 12 having an upstream portion 14 and a downstream portion 16 wherein a fluid flows from the upstream portion to the downstream portion. In some embodiments, crystal growth occurs in primary fluidic channel 12. The phrase “crystal growth” would be understood by one of ordinary skill in the art, and is generally used to refer to the addition of material (e.g., crystal precursor) to a crystal. The additional material may be formed on the crystal such that it at least partially conforms, allowing for crystal defects, to the crystal lattice of the underlying solid. The crystal growth within a fluidic channel may be continuous, which is to say, crystal growth may occur while the crystal is moving within a fluidic channel (e.g., along the length of the channel). For example, in FIG. 1A, a crystal may grow in size while traveling from region 13A to region 13B in channel 12. In some cases, substantially no nucleation occurs in the primary fluidic channel during crystal growth within the primary fluidic channel. “Nucleation” is also a term understood by one of ordinary skill in the art, and is generally used to refer to the beginning of the formation of a crystal. Nucleation may involve combination of material (e.g., dissolved crystal precursor) at the molecular scale to form a very small crystal, for example. It should be understood that crystals may exist in many forms, including many polymorphs, solvates and hydrates, for a given crystal material.

Device 10 also includes a first channel 18 fluidically connected to primary fluidic channel 12. Materials for use in the crystallization process may be flowed through the primary fluidic channel and first channel 18. For example, in some embodiments, a first fluid containing crystal seeds (e.g., organic crystal seeds) may be flowed into primary fluidic channel 12. The first fluid may be, in some cases, saturated with respect to a species contained within the crystal seed at the conditions within the primary fluidic channel. In some embodiments, the first fluid may comprise a crystal precursor (e.g., an undersaturated solution or suspension of crystal precursor, a saturated solution or suspension of crystal precursor, etc.). A second fluid containing a crystal precursor (e.g., a solution of crystal precursor) may be flowed into first channel 18. The crystal precursor in the second fluid may comprise the same species as the crystal seed in the primary fluidic channel. In some cases, the second fluid may be a saturated solution with respect to the crystal precursor at the conditions within the first channel. The second fluid may, in some embodiments, be a supersaturated solution with respect to the crystal precursor at the conditions within the first channel. The supersaturated solution may be made, for example, by combining a saturated or undersaturated solution with antisolvent or nonsolvent, as described below. The second fluid may be delivered to the primary fluidic channel at first feed inlet 20 to the primary fluidic channel. The first and second fluids may be combined to form a first mixed fluid. In some embodiments, the first mixed fluid may remain in a microfluidic channel after mixing. In some embodiments, crystal growth may occur upon the combination of the first and second fluids to form a mixed fluid.

While the first and second fluids are shown as being combined at a T-junction in FIG. 1A, it should be understood that other types of flow systems are suitable for combining any two or more fluids described herein. Examples of different types of flow that can be used include, but are not limited to, multiphase flow (e.g., slug flow, bubbling flow), annular flow, etc. One of ordinary skill in the art will be able to select an appropriate flow scheme for a given application.

A variety of crystal seeds are suitable for use in the embodiments described herein. In some embodiments, the crystal seeds may comprise one material, while the crystal precursor comprises another material (e.g., as in heterogeneous crystal growth). In some cases, crystal seeds may be of one morphology, while the crystal precursor, which comprises the same material as the crystal seeds, forms another morphology on top of the seed due to one or more conditions in which growth occurs. In some embodiments, the crystal seeds are nucleated forms of the crystal precursor contained in one or more of the fluids fed to the primary channel. Crystal seeds may comprise a single species or multiple species (e.g., a co-crystal). In some cases, the crystal seeds may be substantially organic, substantially inorganic, or a co-crystal of at least one organic and at least one inorganic species. A “crystal precursor” refers to any species that forms on a crystal (e.g., to achieve crystal growth) upon combination with the crystal. In some cases, the crystal precursor may comprise a substantially similar material as the crystal on which it is formed. Crystal precursors may be, for example, suspended (e.g., proteins) or dissolved (e.g., ions) in a fluid. Crystal precursors may be organic or inorganic.

Examples of materials suitable for use as crystal seeds or crystal precursors include, but are not limited to pharmaceuticals (e.g. ibuprofen, celcoxib, rofecoxib, valdecoxib, naproxen, meloxicam, aspirin, diclofenac, hydrocodone, propoxyphene, oxycodone, codeine, tramadol, fentanyl, morphine, meperidine, cyclobenzaprine, carisoprodol, metaxalone, chlorpheniramine, promethazine, methocarbamol, gabapentin, clonazepam, valproic acid, phenytoin, diazepam, topiramate, sumatriptan, lamotrigine, oxcarbanepine, phenobarbital, sertraline, paroxetine, fluoxetine, venlafaxine, citalopram, bupropion, amitriptyline, escitalopram, trazodone, mirtanapine, zolpidem, risperidone, olanzapine, quetiapine, promethazine, meclizine, metoclopramide, hydroxyzine, zaleplon, alprazolam, lorazepam, amphetamine, methylphenidate, temazepam, donepexil, atomoxetine, buspirone, lithium carbonate, carbidopa, amoxicillin, cephalexin, penicillin, cefdinir, cefprozil, cefuroxime, ceftriaxone, vancomycin, clindamycin, azithromycin, ciprofloxacin, levofloxacin, trimethoprim, clarithromycin, nitrofurantoin, doxycycline, moxifloxicin, gatifloxacin, tetracycline, erythromycin, fluconazole, valacyclovir, terbinafine, metronidazole, acyclovir, amphotericin, metformin, glipizide, pioglitazone, glyburide, rosiglitazone, glimepiride, metformin, octreotide, glucagon, insulin, human insulin NPH, glargine (insulin), lispro (insulin), aspart (insulin), levothyroxine, prednisone, allopurinol, methylprednisolone, liothyronine, somatropin, colchicine, sulfamerazine, lovastatin, caffeine, cholesterol, lidocaine, strimasterol, theophyllin, acetaminophen, albumin, sporanic acid, lysozyme, mefenamic acid, paracetamol, salmeterol xinafoate, salbutamol, tetracycline or derivatives or parents of the above-mentioned compounds), protein drugs (e.g. interferon, leuprolide, infliximab, trastuzumab, filgastrim, goserelin etc.) pigments (e.g., bronze red, quinacridone etc.), small organic molecules (e.g. glycine, glutamic acid, methionine, flufenamic acid etc.), explosives (e.g. cyclotrimetylenetri-nitramine, nitroguanidine etc.).

In some embodiments, the crystallization rate within a channel may change while fluid is flowed through the channel. Changes in crystallization rate may comprise, for example, a change from substantially no growth to growth, growth to substantially no growth, or a change from a first growth rate to a second growth rate. Changes in crystallization rate may also comprise, for example, a change from substantially no dissolution to dissolution, dissolution to substantially no dissolution, or a change from a first dissolution rate to a second dissolution rate. Changes in crystallization rate may occur, for example, upon changing a condition for crystallization (e.g., a condition within a microfluidic channel). Examples of conditions for crystallization that may be changed include, for example, the temperature, pressure, pH, or composition of a fluid (e.g., type of solvent, type of solute, concentration of solute, type and/or concentration of impurities, etc.) in a channel (e.g., the primary fluidic channel, one or more channels connected to the primary fluidic channel, etc.). Additionally, the temperature of the material from which the channel is fabricated may be changed to change the rate of crystal formation. As a specific example, in some cases, changes in the rate of crystal growth (or dissolution) may occur after the temperature of the fluid within the primary fluidic channel is reduced. As another example, the rate of crystallization may change upon introducing a fluid supersaturated with a crystal precursor into the primary channel containing crystal seeds.

Crystallization rates may also be changed by altering the relative amount of fluid flowed from one or more channels into the primary fluidic channel. For example, in some embodiments, the amount of fluid flowed into the primary fluidic channel from a first channel may be increased relative to the amount of fluid entering the primary fluidic channel, thus resulting in a change in crystallization rate within the primary fluidic channel. In some embodiments, at least one property of the two fluids is different, and, upon mixing the two fluids, at least one property of the mixed fluid is different relative to the same property within the two fluids that are mixed. For example, the first fluid may contain a first concentration of crystal precursor, and the second fluid may contain a second concentration of crystal precursor. Upon mixing, the first and second fluids may form a mixed fluid containing a third, intermediate concentration of crystal precursor. As another example, two fluid streams at different temperatures may be mixed to form a mixed fluid stream at an intermediate temperature.

In some embodiments, multiple channels are connected to primary fluidic channel 12. For example, in FIG. 1A, a second fluidic channel 22 is fluidically connected to primary fluidic channel 12. A fluid containing materials for use in the crystallization process (e.g., a solution of crystal precursor in any suitable state of saturation) may be flowed through the second channel and delivered to the primary fluidic channel at second feed inlet 24 to the primary fluidic channel to form a second mixed fluid. In some instances, the fluid flowed through the second channel may be the same as the fluid flowed through the first channel. For example, in some embodiments, a first fluid containing organic crystal precursor may be flowed through first channel 18 and second channel 22. The first fluid may be flowed into the primary fluidic channel at the first feed inlet 20 and the second feed inlet 24 downstream of the first feed inlet.

In some embodiments, the fluids in the first and second channels may be in the substantially same state of saturation (e.g., undersaturated, saturated, or supersaturated) at the conditions within their respective channels. In other embodiments, the fluids in the first and second channels may be in substantially different states of saturation.

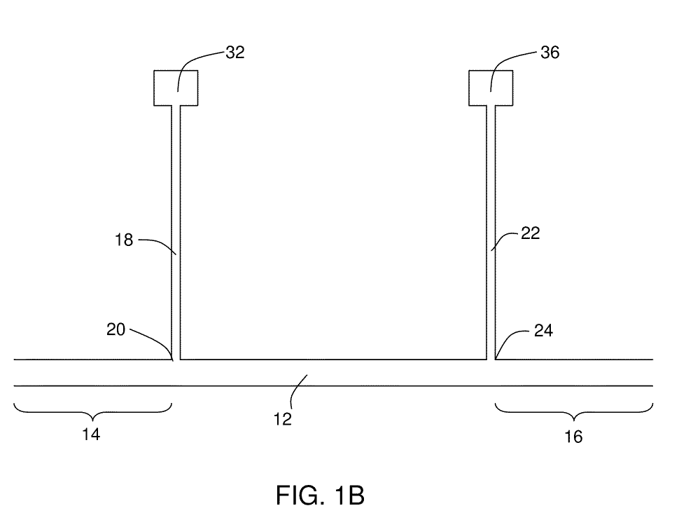

First feed inlet 20 and second feed inlet 24 may be spaced apart by any suitable length, as measured along the length of the primary fluidic channel. For example, in some embodiments the ratio of the distance between the first and second feed inlets, as measured along the length of the primary channel, and the average cross-sectional dimension of the primary channel between the first and second feed inlets is at least about 1:1, 2:1, 3:1, 5:1, 10:1, 25:1, 50:1, 100:1, or greater. It should be noted that the distance as measured along the length of the channel is not necessarily equivalent to the absolute distance between the feed inlets. For example, when multiple feed inlets are positioned along a serpentine channel, the feed inlets may be positioned on subsequent turns of the channel, as illustrated by inlets 20 and 24 in FIG. 2. Although the absolute distance between the two feed inlets is relatively short (indicated by dimension 150), the second feed inlet is positioned a distance along the length of the channel on the order of two passes (indicated by line 152).

In some embodiments, devices may include third, fourth, fifth, or more channels connected to the primary fluidic channel via third, fourth, fifth, or more feed inlets to the primary fluidic channel. These additional channels may be spaced in any suitable fashion. For example, in some embodiments, the channels may be evenly spaced along the length of the primary fluidic channel. In some instances, the spacing between the channels may increase or decrease along the length of the primary fluidic channel. Additional channels connected to the primary fluidic channel may contain the same or different fluid than those contained in the first and/or second channels.

In some embodiments, two or more channels may originate from a common point or region (e.g., a channel, a mixing region, etc.). For example, channels 18 and 22 in FIG. 1A originate from upstream channel 26. When operated in this configuration, a fluid of substantially uniform composition may be added to the primary channel in multiple locations. For example, the concentration of crystal precursor in the fluid within channel 18 may be substantially equal to the concentration of crystal precursor in the fluid within channel 22. Such operation may be useful, for example, in maintaining the concentration of crystal precursor within a range along the length of the primary fluidic channel. As a specific example, a supersaturated solution of crystal precursor may be mixed in the mixing zone and fed to multiple locations along the primary fluidic channel via multiple channels downstream of the mixing zone. As crystal precursor deposits on one or more crystal seeds between first and second feed inlets to the primary fluidic channel, the concentration of the crystal precursor decreases. The supersaturated solution flowed through the second channel may serve to replenish the primary channel with crystal precursor, allowing crystallization to continue. A similar process may be repeated using third, fourth, fifth, and subsequent channels.

The use of multiple feed inlets may allow for control over the concentration of crystal precursor within the primary fluidic channel. For example, in some embodiments, no part of the primary fluidic channel contains a fluid with a crystal precursor concentration of more than about 5 times the solubility limit (i.e., a supersaturation of 5), more than about 2 times the solubility limit, or more than about 1.5 times the solubility limit. In some embodiments, the concentration of crystal precursor in the fluid within the primary fluidic channel is maintained within a range. For example, in some embodiments, the average cross-sectional concentration of the crystal precursor in the primary fluidic channel is between about 1 and about 5 times the solubility limit, between about 1 and about 3 times the solubility limit, or between about 1 and about 2 times the solubility limit at substantially every cross-section between the first inlet feed and the third inlet feed (or between the first and fourth, first and fifth, or first and Nth inlet feeds). As used herein, the “average cross-sectional concentration” is the concentration of a species averaged over the cross-sectional area of a channel, where the cross-sectional area is substantially perpendicular to fluid flow. In some embodiments, the average cross-sectional concentration of the crystal precursor in the primary fluidic channel is between about 1 and about 5 times the solubility limit, between about 1 and about 3 times the solubility limit, or between about 1 and about 2 times the solubility limit at substantially every point between the second inlet feed and the third inlet feed (or between the second and fourth, second and fifth, or second and Nth inlet feeds).

In some embodiments, the concentration of the crystal precursor may be determined in terms of the metastable limit. The metastable limit of a solution generally refers to the concentration above which nucleation occurs. One of ordinary skill would be able to determine the metastable limit for a given solvent and crystal precursor combination. In some embodiments, no part of the primary fluidic channel contains a fluid with a crystal precursor concentration greater than the metastable limit. In some cases, the average cross-sectional concentration of the crystal precursor in the primary fluidic channel is between about 0.2 and about 1 time the metastable limit, between about 0.5 and about 1 time the metastable limit, between about 0.75 and about 1 time the metastable limit, or between about 0.9 and about 1 time the metastable limit at substantially every cross-section between the first inlet feed and the third inlet feed (or between the first and fourth, first and fifth, or first and Nth inlet feeds). In some embodiments, the average cross-sectional concentration of the crystal precursor in the primary fluidic channel is between about 0.2 and about 1 time the metastable limit, between about 0.5 and about 1 time the metastable limit, between about 0.75 and about 1 time the metastable limit, or between about 0.9 and about 1 time the metastable limit at substantially every point between the second inlet feed and the third inlet feed (or between the second and fourth, second and fifth, or second and Nth inlet feeds).

In some cases, two or more channels connected to the primary fluidic channel may not intersect. For example, as shown in FIG. 1B, channels 18 and 22 do not intersect, and their contents originate from unique sources. When operated in this configuration, fluids of substantially same or different composition may be added to the primary channel in multiple locations. This configuration may allow one to vary the concentration and/or composition of the fluid within the primary channel. For example, in some embodiments, the concentration of crystal precursor in the fluid within channel 18 in FIG. 1B may be substantially different than (e.g., less than or greater than) the concentration of crystal precursor in the fluid within channel 22. Operating in this mode allows for the continuous collection of data regarding the effects of variations of one or more conditions for crystallization on crystal growth. As a specific example, the concentration of crystal precursor within a first channel may be 1.5 times the solubility limit, while the concentration of the crystal precursor within the second channel may be 2.5 times the solubility limit. During operation, the rates of crystal growth may be determined between the first and second channels and directly downstream of the second channel. The effect of increasing the concentration of the crystal precursor may be determined during continuous operation, without the need to stop the system and reload seeds and fluids.

In some embodiments, multiple fluid sources may be mixed in the device. For example, as shown in FIG. 1A, the device may include feed section 28 including a first source inlet 30 connectable to a first fluid source 32, and a second source inlet 34 connectable to a second fluid source 36. Any suitable fluid source may be used in the embodiments described herein. For example, a fluid source may comprise a fluidic mixer positioned upstream of a source inlet. In some embodiments, a fluid source may comprise a syringe. The fluid from the first and second sources may be mixed in mixing region 38, which is in fluid communication with the first and second source inlets 30 and 34, respectively.

In one specific example, the first fluid source may contain a under-saturated, saturated, or supersaturated solution of crystal precursor, and the second fluid source may contain an antisolvent. The solution of crystal precursor and the antisolvent may be flowed into a mixing region, where the two are mixed to form a supersaturated solution with a higher concentration of crystal precursor than the concentration in the first fluid. In some embodiments, the antisolvent should be selected such that the antisolvent is soluble in the solvent of the crystal precursor solution, but the crystal precursor is insoluble in the antisolvent. As a specific example, ethanol or acetone may be used as the antisolvent to produce a supersaturated solution of glycine in water. Those skilled in the art will know of suitable antisolvents, or will be able to ascertain such, using only routine experimentation.

In some embodiments, the mixed fluid may remain flowing within a microfluidic channel. In some embodiments, the length of the microfluidic channel through which the mixed fluid is flowed may be at least about 2 times, at least about 5 times, at least about 10 times, at least about 25 times, at least about 50 times, at least about 100, at least about 1,000, or at least about 10,000 times the largest cross sectional dimension of the microfluidic channel at the point of mixing.

Devices may include, in some instances, third, fourth, fifth, or more source inlets. One or more additional source inlets may be connectible to one or more additional fluid sources. In some embodiments, one or more additional source inlets may be connected to the same fluid sources as one or both of the first two fluid sources. For example, in one set of embodiments, first and third source inlets may be connected to the same fluid source, while the second source inlet is connected to a different fluid source. By positioning the first and third source inlets on either side of the second source inlet, diffusional mixing of the two fluids may be improved within the mixing region.

In some embodiments, a fluid containing an organic crystal and a crystal precursor is flowed through a channel, and one or more properties of the crystal may be determined in at least one location. In some embodiments, a fluid containing crystal precursor may contain a plurality of crystals, and one or more properties of two or more crystals may be determined. Examples of properties of a crystal that may determined include, but are not limited to, a dimension (e.g., diameter, longest dimension, length, distances between crystal planes, or any other dimension), shape, one or more angles between crystal planes, and crystallographic orientation (e.g., morphology of a single crystal, morphologies of multiple crystals in a co-crystal, morphologies of multiple crystals in a collection of separate crystals, etc.), materials of composition, among others. In some embodiments, the morphologic composition of a single crystal (i.e., the percentage (e.g., weight percentage) of each morphology type within a single crystal) may be determined.

In some embodiments, a property (e.g., a dimension) of each of a plurality of crystals may be determined, which may be used to determine a property of the plurality of crystals (e.g., size distribution, morphology distribution, etc.). For example, in some embodiments, the morphologic composition of a plurality of crystals may be determined. The morphologic composition of the collection of crystals may be determined by calculating the relative amounts of each morphology type among a collection of crystals. For example, if 10 crystals are present, 4 with a first morphology and 6 with a second morphology, the morphologic composition, by number, would be 40% for the first morphology and 60% for the second morphology. Morphologic composition may also be calculated, in some cases, on a mass basis. It should be noted that the morphologic composition of a plurality of crystals can also be calculated when one or more crystals comprises multiple crystal morphologies. For example, if 10 crystals of equal mass are present, 4 with a 50%/50% (by mass) mix of first and second morphologies and 6 including only the second morphology, the morphologic composition of the plurality of crystals, by mass, would be 20% for the first morphology and 80% for the second morphology.

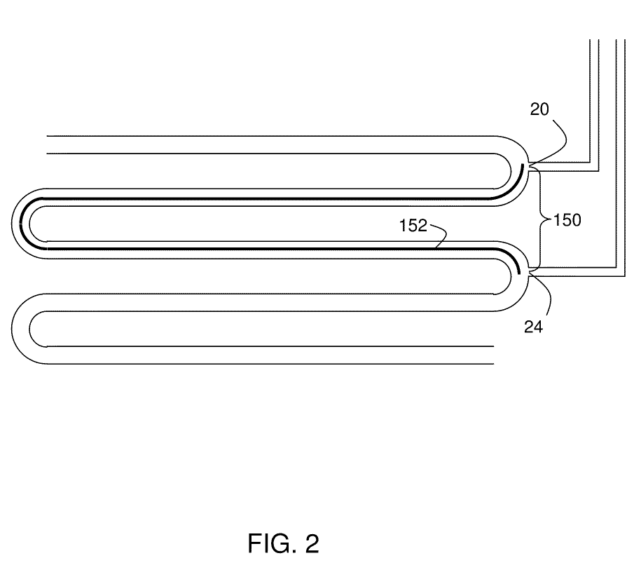

As used herein, the “largest dimension” is measured along the longest line that can be drawn between two exterior points of a crystal. For example, in FIG. 3A, the longest dimension of the crystal is illustrated by dimension 302, whereas the length of the crystal is shown as dimension 304. In some embodiments, the length and largest dimension of the crystal are the same, as shown by dimension 306 in FIG. 3B. The determination of a property of a crystal may be useful, for example, in studying the effects of one or more conditions for crystallization (e.g., temperature, pressure, pH, concentration, etc.) on the dynamics of crystallization within the channel.

In some embodiments, a first property of the crystal is determined at a first point in the fluidic channel, and a second property of the crystal is determined at a second point in the fluidic channel. In some embodiments, the first and second properties may be the same type (e.g., dimension, crystallographic orientation, etc.). For example, the length of the crystal may be determined at a first point in the fluidic channel and at a second point in the fluidic channel, where the length may be substantially the same, shorter, or longer. Determining the length of a crystal at two or more points in a channel may be useful, for example, in calculating the growth rate or dissolution rate of the crystal within the channel. For example, the growth rate or dissolution rate of a crystal may be calculated by dividing the difference in length of the crystal at two different points within the channel by the time spent in the channel. The amount of time a crystal spends in a continuous flow device may be calculated, for example, by dividing the length the crystal travels by the linear velocity of the crystal. Thus, accurate growth rate and kinetics measurements may be made using image analysis. In some cases, accurate growth rate and kinetics measurements may be made without using a timing device, if the flow rate of the fluid and the relationship between the flow rate of the fluid and the flow rate of the crystals is known.

The first property determined at the first point in the channel, in some embodiments, may be a different type than the second property determined at the second point. For example, the length of the crystal may be determined at a first point in the channel while the crystallographic orientation may be determined at a second point in the channel. Such an arrangement may be used, for example, when different apparatuses are required to determine the first and second properties.

In some embodiments, at least one property of a crystal, comprising a species, is determined in a channel, and, based upon the crystal determination step, at least one condition for crystallization of the species is determined. Examples of conditions for crystallization that may be determined include, for example, a temperature of a fluid or channel, a pressure within a channel, the concentration and/or composition of a species within a fluid, the flow rate of one or more fluids (which may determine, for example, the residence time of a crystal in a channel), the pH of a fluid, and the like. Once the condition for crystallization has been identified, some embodiments may further comprise growing crystals comprising the species involving at least the condition. For example, in some embodiments, the crystallographic orientation of a crystal may be determined in a channel operated at a temperature. It may be determined, based at least in part upon the determined crystallographic orientation, that the temperature of the channel produces crystals with a particularly desirable crystal morphology. The temperature may be used in subsequent crystal growth processes (e.g., experimental process, industrial production processes, etc.). As another example, the growth rate of a crystal may be determined in a channel operated at a temperature. The temperature may be used in subsequent crystal growth processes to achieve the desired growth rate.

The term “determining,” as used herein, generally refers to the analysis or measurement of a species (e.g., a crystal, a crystal precursor, a fluid, an impurity etc.), a property (e.g., a dimension, crystallographic orientation, morphology, etc.), or condition (e.g., flow rate, temperature, pressure, pH, evaporation rate, etc.), for example, quantitatively or qualitatively, and/or the detection of the presence or absence of the species, property, or condition. “Determining” may also refer to the analysis or measurement of an interaction between two or more species, two or more properties, two or more conditions, or between a combination of two or more species, properties, and conditions, for example, quantitatively or qualitatively, or by detecting the presence or absence of the interaction. For example, determining may comprise measuring the effect of a change in a channel dimension on crystal morphology of one or more crystals. Examples of suitable techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical microscopy or optical density measurements; circular dichroism; light scattering measurements such as quasielectric light scattering; polarimetry; refractometry; or turbidity measurements. In some embodiments, at least a portion of the device in which crystallization occurs is transparent to at least one wavelength of electromagnetic radiation (e.g., x-rays, ultraviolet, visible, IR, etc.) allowing interrogation of, for example, the crystal, the channel, the fluid, etc. For example, optical microscopy may be used to determine one or more crystal properties such as a dimension, shape, the presence or absence of a crystal, etc. The systems used to determine a property of the crystals may be interfaced with a computer to allow for real-time analysis. For example, images of crystals may be analyzed in real time using image analysis software. This may allow for on board for real-time determination of reaction kinetics, which may be used in subsequent reaction runs to optimize the crystallization process. In addition, real time analysis may allow integration of feedback control loop, enabling one to achieve crystals with the desired property (e.g. size, size distribution, morphology, morphologic distribution, etc.).

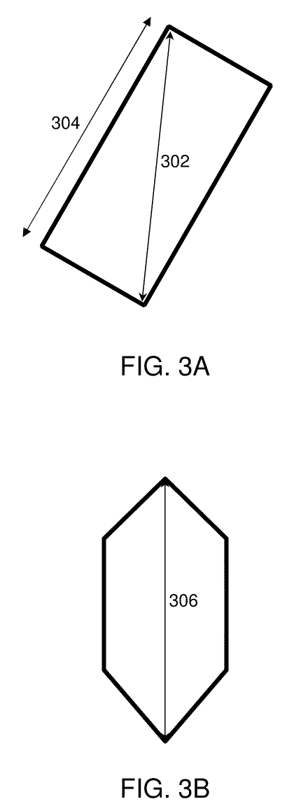

When transported in laminar flow, the lengths of the crystals may align in a direction substantially perpendicular to the flow of fluid. For example, FIG. 4 is an image of a primary channel 12 in which crystals 402 are substantially aligned with the flow of fluid within the channel. Not wishing to be bound by any theory, the lengths of the crystals may align in order to minimize the shear on the crystal created by continuous flow. Some embodiments comprise flowing a fluid comprising crystals (or other particles, as discussed below) with aspect ratios of at least about 3:1, at least about 10:1, at least about 25:1, or at least about 50:1 and a crystal precursor (or other particle precursor) within a microfluidic channel. As the crystals are flowed through the microfluidic channel, they may become substantially aligned within the channel. After the crystals are substantially aligned (e.g., in a direction substantially perpendicular to the flow of fluid) a first property of the crystal may be determined at a first point in the microfluidic channel and a second property of the crystal may be determined at a second point in the microfluidic channel.

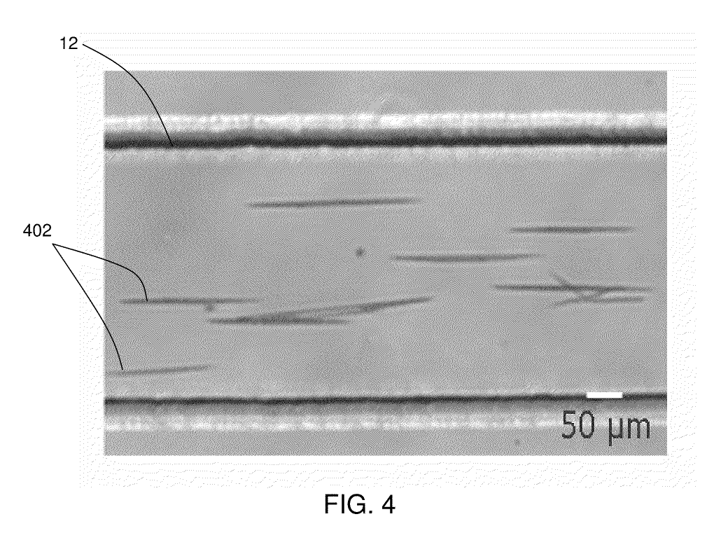

Accurate measurement of crystal length may be easier when the crystal is interrogated from an angle substantially perpendicular to crystal length. Thus, the alignment of the crystals in the direction of fluid flow can aid in the measurement of crystal dimensions in some embodiments. To illustrate, in one commonly-used crystal size measurement, a Lasentech probe directs light onto the crystal, which is reflected back to the probe and measured. This process is illustrated, for example, in FIGS. 5A-5B. FIG. 5A includes a schematic illustration of a beaker containing multiple crystals suspended in liquid. The crystals in FIG. 5A are being interrogated by the focused beam of a Lasentech probe from the top side of the beaker. FIG. 5B includes a cross-sectional top view of the beaker and the crystals within it. The section of the liquid within the beaker that is scanned by the Lasentech probe is illustrated by shaded pathway 502 in FIG. 5B. FIG. 5C illustrates the crystal size distribution as measured by the Lasentech probe, while FIG. 5D illustrates the actual crystal size distribution. As seen from these figures, failure to interrogate the crystals at an angle perpendicular to the crystal length has produced inaccuracies in the measured length in FIGS. 5A-5D. Specifically, the crystal lengths measured by the Lasentech probe in FIG. 5C are shorter than the actual lengths illustrated in FIG. 5D. This effect may be pronounced for acicular crystals (e.g., needles) and other high aspect ratio crystals. When crystals are aligned, perpendicular interrogation is substantially easier to achieve.

Embodiments are described generally herein with reference to crystallization in microfluidic channels. It should be understood, however, that the invention is not limited to the use of crystals, and in all locations herein in which crystal formation and/or growth is described, in alternative embodiments amorphous particles (e.g., particles that comprise amorphous portions, particles that are substantially amorphous, etc.) can be produced. For example, the alignment of relatively high aspect ratio particles in a microfluidic stream may be useful in measuring changes in the size of both amorphous and crystalline particles. Generally, when amorphous particles are grown, the material that is deposited onto the particle is referred to as particle precursor, rather than crystal precursor. The amorphous particle formed in such embodiments may have properties similar to those of the crystalline particles (e.g., composition, size, shape, etc.), with the exception of the difference in crystallinity. Those of ordinary skill in the art will recognize how, and with which materials, features of the present invention described herein with respect to crystalline material, can be applied to non-crystalline materials. Those of ordinary skill in the art will be familiar with classes of materials suitable for forming elongated amorphous structures. Some examples of organic molecules that exhibit anisotropic structural properties in their solid forms, giving rise to acicular or needle-shaped crystals are the beta form of glycine, lovastatin, some polymorphs of ROY (5-methyl-2-[(2-nitrophenyl)amino]-3-thiophenecarbonitrile) such as ON and YN, irbesartan (an API), hydroquinone etc. Examples of inorganic materials that may give rise to high aspect ratio particles include barium titania (BaTiO₃,) titanium oxide, iron (β-FeOOH), hydrogoethite (α-FeOOH.xH₂O), manganese-zinc ferrite, BaSn(OH)ₓ, alumina, zirconia, etc. Examples of materials that may give rise to acicular amorphous particles are iron oxide, silica, titania, iron-cobalt etc.

As used herein, the term “fluid” generally refers to a substance that tends to flow and to conform to the outline of its container. Typically, fluids are materials that are unable to withstand a static shear stress, and when a shear stress is applied, the fluid experiences a continuing and permanent distortion. The fluid may have any suitable viscosity that permits at least some flow of the fluid. Non-limiting examples of fluids include liquids, gases, and supercritical fluids, but may also include free-flowing solid particles (e.g., colloids, vesicles, etc.), viscoelastic fluids, and the like.

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more typically at least 3:1, 5:1, or 10:1 or more. The “cross-sectional dimension” of a channel is measured perpendicular to the direction of fluid flow.

The channel may be of any size, for example, having a largest cross-sectional dimension of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flow rate of fluid in the channel. In some embodiments, the length of the channel may be selected such that the residence times of a first and second (or more) fluids at a predetermined flow rate are sufficient to produce crystals of a desired size or morphology. Lengths, widths, depths, or other dimensions of channels may be chosen, in some cases, to produce a desired pressure drop along the length of a channel (e.g., when a fluid of known viscosity will be flowed through one or more channels). Of course, the number of channels and the shape of the channels can be varied by any method known to those of ordinary skill in the art.

In some, but not all embodiments, some or all components of the systems and methods described herein are microfluidic. “Microfluidic,” as used herein, refers to a device, apparatus or system including at least one fluid channel having a largest cross-sectional dimension of less than about 1 mm, and a ratio of length to largest cross-sectional dimension perpendicular to the channel of at least 3:1. A “microfluidic channel” or a “microchannel” as used herein, is a channel meeting these criteria. In one set of embodiments, all fluid channels containing embodiments of the invention are microfluidic.

A variety of materials and methods, according to certain aspects of the invention, can be used to form systems such as those described above. In some embodiments, the channel materials are selected such that the interaction between channel surfaces and crystals and/or crystal precursor materials is minimized. Minimizing such interactions may assist in reducing the amount of crystal nucleation in the channel (e.g., on channel walls), as well as adhesion/interaction of crystals on/with the channel walls, thus minimizing channel clogging. For example, when crystals and/or crystal precursors comprise charged particles, the channel material may be selected such that the charged materials are repelled from the channel surface. In some cases, one or more channel surface portions may be coated with a material that serves to minimize the interactions between the channel surface portion(s) and the crystals and/or crystal precursor materials within the channel. For example, channels may be coated with a hydrophobic material to repel, for example, water-soluble particles. Similarly, channels may be coated, in some embodiments, with hydrophilic material to repel, for example, water-insoluble particles. For example, glycine, which exists in zwitterionic form in solid states, is relatively hydrophilic and is repelled by hydrophobic materials/coatings such as polydimethylsiloxane, or fluorosilane. As another example, aspirin, a water-insoluble drug, comprises hydrophobic groups at its crystal planes, and is repeled by hydrophilic materials or coatings such as glass, silicon, and silanes with hydrophilic groups.

In some embodiments, the fluid channels may comprise tubing such as, for example, flexible tubes (e.g., PEEK tubing), capillary tubes (e.g., glass capillary tubes), and the like. In some embodiments, various components can be formed from solid materials, in which microfluidic channels can be formed via micromachining, film deposition processes such as spin coating and chemical vapor deposition, laser fabrication, photolithographic techniques, soft lithographic techniques, etching methods including wet chemical or plasma processes, and the like. See, for example, Scientific American, 248:44-55, 1983 (Angell, et al). In one set of embodiments, at least a portion of the fluidic system is formed of silicon by etching features in a silicon chip. Enclosed channels may be formed, for example, by bonding a layer of material (e.g., polymer, Pyrex®, etc.) over the etched channels in the silicon. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®) or rigid polymers such as poly(methyl methacrylate) (PMMA), cyclic olefin copolymer (COC) (e.g. TOPAS), or the like. In some cases, various components of the system may be formed in other materials such as metal, ceramic, glass, Pyrex®, etc. In some embodiments, various components of the system may be formed of composites of these materials herein.

Different components can be fabricated of different materials. For example, a base portion including a bottom wall and side walls can be fabricated from a transparent or at least partially transparent material, such as glass or a transparent polymer, for observation and/or control of the fluidic process, and a top portion can be fabricated from an opaque material such as silicon. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In one embodiment, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point/glass transition temperature. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are preferred in one set of embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 85° C. for exposure times of, for example, about two hours. Also, silicone polymers, such as PDMS, can be elastomeric, and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic channel surfaces can thus be more easily filled and wetted with aqueous solutions.

In one embodiment, a bottom wall of a microfluidic device of the invention is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, bonding, solvent bonding, ultrasonic welding, etc.

The following examples are intended to illustrate certain embodiments of the present invention, but do not exemplify the full scope of the invention.

### EXAMPLE 1

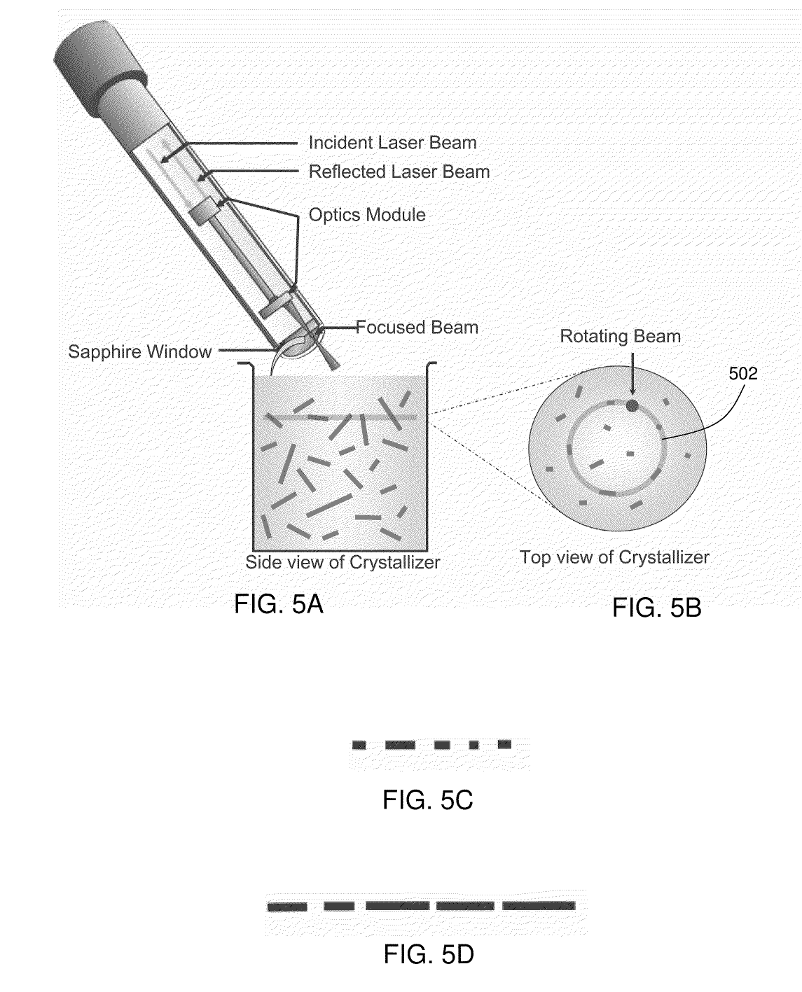

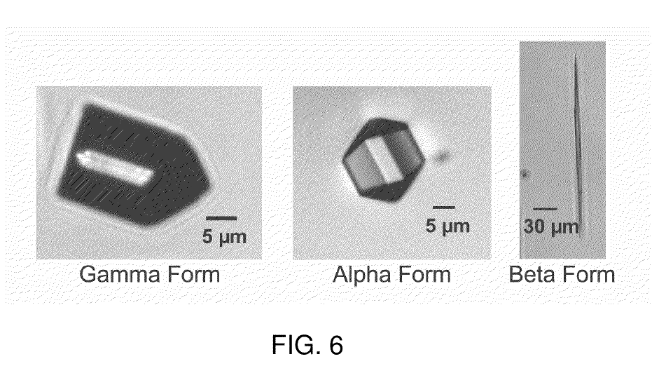

This example describes crystallization of glycine, according to one set of embodiments. The three most common polymorphs of glycine—gamma, alpha and beta—are shown in FIG. 6. Seeds of each of the three glycine polymorphs were introduced into the reactor in separate experiments, and their growth rates were calculated. In this example, seeded crystallization was used to eliminate uncontrolled nucleation. FIG. 7 includes a schematic illustration of the reactor used in this example. In one inlet, a feed of saturated aqueous glycine seeds was delivered at 10 microliters/min to the microfluidic device at room temperature. Via another inlet, a 40% saturated aqueous glycine solution was fed to the device. In a third inlet, an antisolvent of pure ethanol was fed to the device. The antisolvent and the 40% saturated aqueous glycine solution were mixed on chip to generate a supersaturated solution of glycine. The supersaturated glycine solution was subsequently added to the primary fluidic channel in small, controlled quantities. The glycine precursors added to the primary fluidic channel grew on the glycine seed crystals, and the crystals increased in size. The flow rates of the crystallizing solution, antisolvent and seed flowrates were selected such that the supersaturation was kept below the nucleation regime at all times inside the device, thus eliminating undesired secondary nucleation in the system. Moreover, spurious heterogeneous nucleation on the reactor walls was limited by using an inert reactor, controlling impurities inside the primary fluidic channel, and limiting the formation of gas within the primary fluidic channel. By eliminating secondary nucleation within the device, unwanted clogging of the channels was avoided.

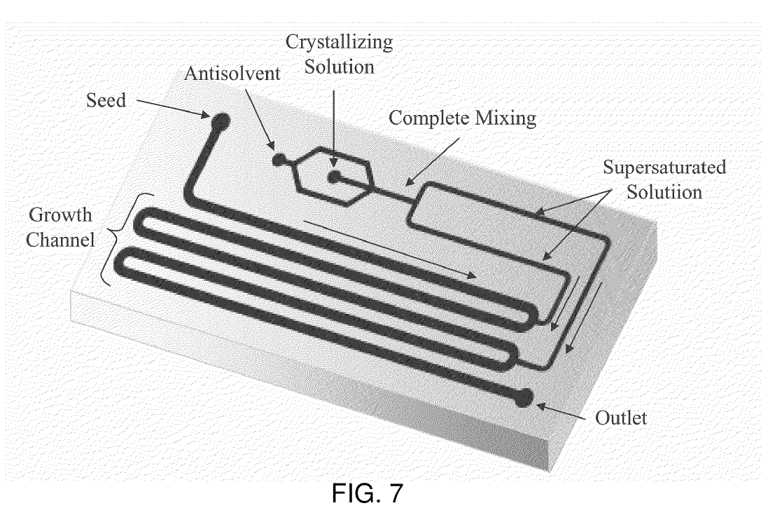

The growth rates of alpha, beta, and gamma glycine were calculated by capturing optical images of the channel at two different times and measuring the difference in the crystal length. A Matlab code and Adobe Photoshop were used to analyze the size distribution of the crystals. The growth rates of different planes were calculated from the growth rates of the length and width using the angles between the planes' linear dimensions. For example, the growth rate of planes <011> and <010> in alpha glycine were calculated from the change in L_{b} and L_{c} (illustrated in FIG. 8) as follows:

$${G}_{( 011 )} = \frac{1}{2} sin {67.5}^{o} \frac{d {L}_{c}}{d t}$$

$${G}_{( 010 )} = \frac{1}{2} sin {68.5}^{o} \frac{d {L}_{b}}{d t}$$

The growth rates are presented in Table 1, Table 2, and Table 3, respectively. The three polymorphs have widely varied shapes, and the effectiveness with which their growth rates were measured demonstrates the versatility of the technique. To validate the growth rates obtained in the microfluidic devices, the growth rate of the alpha form was calculated with suggested parameters from the literature and compared with the experimental values. As shown in Table 1, the experimental values were well within the predicted values.

TABLE 1
Experimental (Exp) and Predicted (Pred) Growth Rates of
the <011> and <010> Crystal Planes of α-Glycine

| | G_{<011>, exp} | G_{<011>,pred} | G_{<010>,exp} | G_{<010>,pred} |
|---|---|---|---|---|
| S = ln(C/C_{s,α}) | (μm/min) | (μm/min) | (μm/min) | (μm/min) |
| 0.33 | 2.22 ± 0.91 | 2.17 ± .75 | 0.40 ± 0.25 | 0.28 ± 0.32 |
| 0.56 | 3.34 ± 0.43 | 3.74 ± 1.29 | 0.74 ± 0.16 | 0.47 ± .53 |
| 0.64 | 4.78 ± 1.35 | 4.29 ± 1.48 | 0.66 ± 0.23 | 0.56 ± 0.63 |

Note:
The predicted growth rates were calculated using values from L. Li, N.R.-H., Growth kinetics and mechanism of glycine crystals. Journal of Crystal Growth, 1992. 121: p. 33-38.

TABLE 2
Experimental Growth Rates of
β-Glycine

| S = ln(C/C_{s,α}) | G_{<010>} (μm/min) |
|---|---|
| 0.30 | 194 ± 55 |
| 0.39 | 235 ± 43 |
| 0.47 | 250 ± 52 |

TABLE 3
Experimental Growth Rates of
γ-Glycine

| Supersaturation, | G_{{100, 010},exp} | G_{<00-1>,exp} |
|---|---|---|
| S = ln(C/C_{s,γ}) | (μm/min) | (μm/min) |
| 0.319 | 3.2 ± 1.6 | 4.4 ± 1.8 |
| 0.389 | 5.5 ± 1.6 | |

### EXAMPLE 2

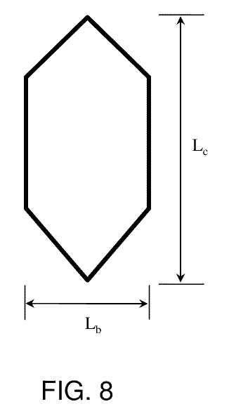

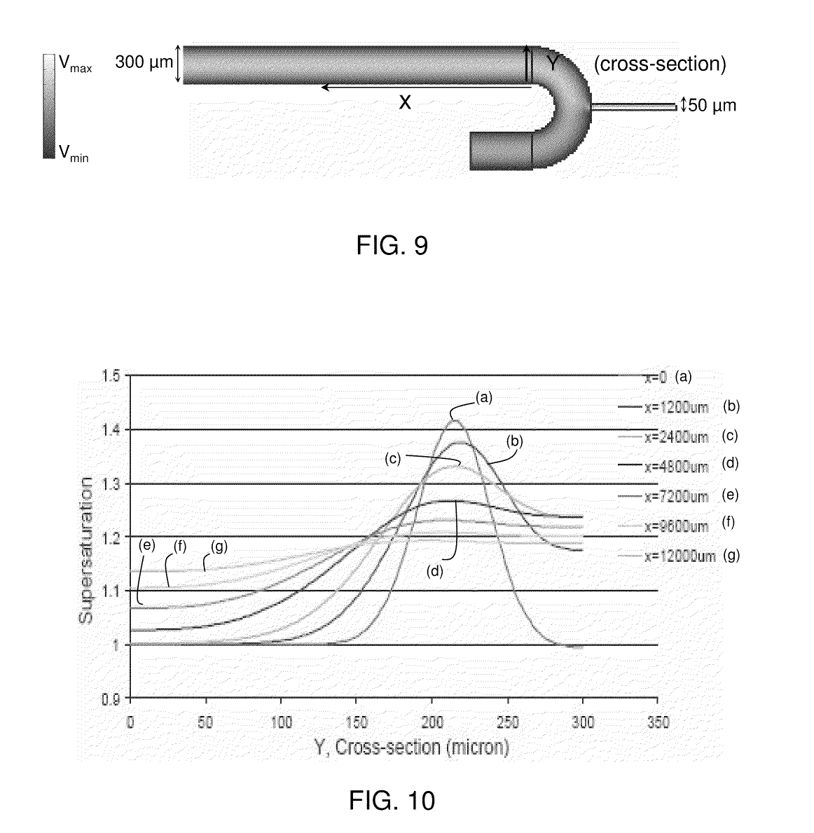

In this example, simulations were performed to study the transport of fluid and crystal precursor within a microfluidic channel. FIG. 9 illustrates the fluid velocity profile of a primary fluidic stream mixed with a side-stream. The velocity profile of this aqueous solution was simulated using FEMLAB, a multiphysics modeling and analysis software. FIG. 10 includes calculations of supersaturation as a function of cross-sectional position at various points along the length of the channel. As seen from the plot, substantially uniform supersaturation is achieved 12 mm from the point of mixing. This corresponds to a time of approximately 5 seconds, or about 3.4% of the length of the channel over which growth occurs in this example. The short mixing time was much smaller than the dispersion inherent in the crystal growth process, which is in the order of about 30% for organic crystals, as shown in L. Li, et al., Growth kinetics and mechanim of glycine crystals. Journal of Crystal Growth, 1992. 121: p. 33-38, which is incorporated herein by reference.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of determining crystallization, comprising:
flowing a fluid containing an organic crystal and crystal precursor into a microfluidic channel;
determining a first property of the crystal at a first point in the microfluidic channel; and
determining a second property of the crystal at a second point in the microfluidic channel.

2. The method of claim 1, wherein the fluid contains the organic crystal and a solution of crystal precursor.

3. The method of claim 2, wherein the solution of crystal precursor is a supersaturated solution of crystal precursor.

4. The method of claim 3, wherein the fluid containing the crystal and the supersaturated solution of crystal precursor is formed by combining a first fluid containing an organic crystal seed with a second fluid containing a supersaturated solution of crystal precursor.

5. The method of claim 1, wherein the first or second properties of the crystal are a dimension of the crystal.

6. The method of claim 1, wherein multiple crystals are flowed into the microfluidic channel, and the crystal size distribution of the multiple crystals is determined.

7. The method of claim 1, wherein the first or second properties of the crystal are the shape of the crystal.

8. The method of claim 1, wherein the first or second properties of the crystal are a crystallographic orientation of the crystal.

9. The method of claim 1, further comprising determining the crystal growth rate.

10. The method of claim 1, wherein determining the first or second property comprises optical imaging.

11. The method of claim 1, wherein the first or second properties of the crystal are morphologic composition of the crystal.

12. The method of claim 1, wherein determining the first or second property comprises x-ray crystallography.

13. The method of claim 1, wherein the determining step comprises spectroscopy.

14. The method of claim 1, further comprising changing at least one condition for crystallization.

15. The method of claim 14, wherein the condition for crystallization comprises the temperature of the microfluidic channel.

16. The method of claim 14, wherein the condition for crystallization comprises the concentration of a solute in the microfluidic channel.

17. The method of claim 14, wherein the condition for crystallization comprises the composition of a solute or solvent in the microfluidic channel.

18. The method of claim 14, wherein the condition for crystallization comprises the concentration of impurities within the microfluidic channel.

19. The method of claim 14, wherein the condition for crystallization comprises pH.

20. The method of claim 1, wherein the flow of fluid in the microfluidic channel is laminar.

21. A method of forming crystals, comprising:
flowing a first fluid containing organic crystal seeds into a first microfluidic channel;
flowing a second fluid containing a solution of crystal precursor into a second microfluidic channel; and
combining the first and second fluids to form a first mixed fluid.

22. The method of claim 21, wherein the second fluid contains a supersaturated solution of crystal precursor.

23. The method of claim 21, wherein the first mixed fluid remains in a microfluidic channel.

24. The method of claim 21, further comprising flowing a third fluid containing a supersaturated solution of crystal precursor into a third microfluidic channel, and combining the third fluid and first mixed fluid to form a second mixed fluid.

25. The method of claim 21, wherein, prior to the combining step, substantially none of the crystal seeds grow.

26. The method of claim 21, wherein, subsequent to the combining step, at least one of the crystal seeds grow.

27. The method of claim 24, wherein the concentration of crystal precursor in the second fluid is substantially equal to the concentration of crystal precursor in the third fluid.

28. The method of claim 24, wherein the concentration of crystal precursor in the second fluid is substantially different than the concentration of crystal precursor in the third fluid.

29. A method of forming crystals, comprising:
flowing a first fluid containing organic crystal precursor into a microfluidic channel at a first feed inlet; and
flowing the first fluid containing organic crystal precursor into the microfluidic channel at a second feed inlet downstream of the first feed inlet.

30. The method of claim 29, wherein the ratio of the distance between the first and second feed inlets, as measured along the length of the microfluidic channel, and the average cross-sectional dimension of the microfluidic channel between the first and second inlets is at least about 1:1

31. A method, comprising:
determining at least one property of a crystal, comprising a species, in a microfluidic channel;
based upon the crystal determination step, determining at least one condition for crystallization of the species; and
growing crystals comprising the species involving the at least the condition.

32. The method of claim 31, wherein the condition for crystallization of the species is a temperature.

33. The method of claim 31, wherein the condition for crystallization of the species is pressure.

34. The method of claim 31, wherein the condition for crystallization of the species is evaporation of the solvent.

35. The method of claim 31, wherein the condition for crystallization of the species is the concentration of the species within a fluid.

36. The method of claim 31, wherein the condition for crystallization of the species is the composition of the species within a fluid.

37. The method of claim 31, wherein the condition for crystallization of the species is the pH of a fluid.

38. The method of claim 31, wherein the property comprises a dimension of the crystal.

39. The method of claim 38, further comprising determining a dimension of a plurality of crystals in the microfluidic channel, and determining the size distribution of the plurality of crystals.

40. The method of claim 31, wherein the property comprises the shape of the crystal.

41. The method of claim 31, wherein the property comprises a morphology of the crystal.

42. The method of claim 31, wherein the property comprises morphologic composition of the crystal.

43. A microfluidic device, comprising:
a primary microfluidic channel having an upstream portion and a downstream portion, wherein fluid flows from the upstream portion to the downstream portion;
a feed section including a first source inlet connectable to a first fluid source, a second source inlet connectable to a second fluid source, and a mixing region in fluid communication with the first and second source inlets, at which fluids from the first and second sources are mixed;
a first channel connecting the mixing region with a first feed inlet to the primary microfluidic channel, for delivery of fluid from the mixing region to the primary microfluidic channel; and
a second channel connecting the mixing region with a second feed inlet to the primary microfluidic channel, for delivery of fluid from the mixing region to the primary microfluidic channel.

44. A method of determining particle formation, comprising:
flowing a fluid containing a particle with an aspect ratio of at least about 3:1 and a particle precursor within a microfluidic channel;
determining a first property of the particle at a first point in the microfluidic channel; and
determining a second property of the particle at a second point in the microfluidic channel,
wherein the determining steps are performed after the particle is substantially aligned in the direction of fluid flow within the microfluidic channel.

45. The method of claim 44, wherein the fluid contains a plurality of particles with aspect ratios of at least about 3:1.
